(19) United States
(12) Patent Application Publication
(10) Pub. No.: US 2003/0152515 A1
(43) Pub. Date: Aug. 14, 2003

(54) Method for estimating effective regimens and patient survival rates of antibiotic treatments for fatal infectious diseases

(76) Inventor: Ren-Jin Lee, Gaithersburg, MD (US)

Correspondence Address: Ren-Jin Lee, 10845 Outpost Drive, Gaithersburg, MD, 20878, US

(21) Appl. No.: 10/066,571

(22) Filed: Feb. 6, 2002

(51) Int. Cl. A61K 49/00; G06F 19/00; G01N 33/48; G01N 33/50

(52) U.S. Cl. 424/9.2; 702/19

(57) ABSTRACT

The present invention relates to a method for estimating effective regimens and patient survival rates of antibiotic treatments for fatal infectious diseases. More particularly, the invention estimates the effective dose regimens and the patient survival rate, utilizing (1) the survival data of patients receiving either no treatment or inactive treatments and (2) the correlation between the organism eradication rate and the dose regimen of the antibiotics.

### CROSS-REFERENCE TO RELATED APPLICATIONS

Not applicable.

### BACKGROUND OF THE INVENTION

1. Field of the Invention

The present invention relates to a method for estimating effective regimens and patient survival rates of antibiotic treatments for fatal infectious diseases. More particularly, the invention estimates the effective dose regimens and the patient survival rate, utilizing (1) the survival data of patients receiving either no treatment or inactive treatments and (2) the correlation between the organism eradication rate and the dose regimen of the antibiotics.

2. Description of the Related Art

Many infectious diseases are fatal and require immediate and aggressive treatments for the patients. The dosing regimens of antibiotics for treating the fatal infectious diseases need to be carefully strategized since the consequence of underdosing the patients can be devastative.

The therapeutic response to antibiotic treatments for infectious diseases can be highly variable, influenced by the patient characteristics, such as age and body weight, and the variability in pharmacokinetics and pharmacodynamics of the antibiotics. Therefore, it is crucial to individualize antibiotic dosing regimens for treating fatal infectious diseases.

Previously, there are several approaches in determining the dosing regimens for fatal infectious diseases or infectious diseases in general. The first is a one-size-fits-all approach, in which the same regimen is giving all patients, regardless of their characteristics. An example of the first approach is the regimen of doxycycline and penicillin for treating anthrax [Physician's Desk Reference, 2001]. The second approach is to adjust the dose regimen for individual patients by targeting specific values of a pharmacodynamic marker estimated from population pharmacokinetic models. An example of the second approach is the dosing strategy of ciprofloxacin for lower respiratory infection [Forrest et al, 1993(b)]. The third possible approach is to conduct clinical trials in sub-populations and determine the dose regimens for each of the populations.

There are many drawbacks of the above three approaches. The first one-size-fits-all approach is clearly not suitable for treating fatal infectious diseases using antibiotics with highly variable response rates. The second approach, which targeting specific pharmacodynamic markers, may not be useful, since the linkages between the markers and patient survival rate may not be clearly established. The third approach, in which clinical trials are conducted in sub-populations, may not be practical, as the cost of conducting the studies can be tremendous.

### BRIEF SUMMARY OF THE INVENTION

The present invention seeks to overcome the drawbacks inherent in the prior art by providing new methods for determining the antibiotic regimens for treating fatal infectious diseases.

Therefore, one object of the present invention is to provide a method to estimate the survival rate of a patient population with a fatal infectious disease following an antibiotic treatment, without conducting any clinical trial on the specific patient population.

Another object of the present invention is to provide a method to estimate the effective antibiotic regimens to treat a patient population with a fatal infectious disease without conducting any clinical trial on the specific patient population.

Accordingly, the invention provides methods to estimate the patient survival rate following antibiotic treatments for fatal infectious diseases, based on (1) the survival rate of controlled patients receiving no treatment or inactive treatments, and (2) the correlation between organism eradication rate and antibiotic regimen. The invention also provides methods to estimate the effective antibiotic regimens for treating fatal infectious diseases, based on (1) the survival rate of controlled patients receiving no treatment or inactive treatments, (2) the correlation between organism eradication rate and antibiotic regimen, and (3) the targeted therapeutic effect on the survival rate of the antibiotic-treated patients.

Since the deaths of the patients with a fatal infectious disease are associated with the presence of the infective organisms in the patient's body, it is plausible that patients, antibiotic-treated or controlled, with the same duration of systemic exposure to the infective organism should have a similar mortality rate. In another words, the mortality rate of the antibiotic-treated patients with positive culture is likely to be similar to that of the controlled patients, as long as a significant amount of the infective organisms remain in the patients. This similarity in mortality rate between the antibiotic-treated and the controlled patients is especially plausible when the infectious disease is highly progressive and the fatality occurs within a short period of time after the disease onset.

Therefore, the survival rate of the antibiotic-treated patients can be estimated from the survival rate of the controlled patients, if the duration of exposure to the organism in the former can be determined from the organism eradication rate.

It is important to note that the invention utilizes survival data only from the controlled patients who do not receive the antibiotic. The survival data of the controlled patients can be available from epidemiological studies or, in some rare cases, from prospectively designed studies. Patients with infectious diseases may sometimes, mostly unintentionally, be treated with inactive treatments (including placebo) or may receive no treatment due to various reasons, such as a lack of available medicines. The patient survival rate is usually expressed as a function of time after the disease onset.

The correlation between organism eradication rate and antibiotic regimen can be derived utilizing one of more of the following information: (1) the pharmacodynamic marker of the antibiotic, (2) the pharmacokinetics of the antibiotic, and (3) the characteristics of the patients;

The organism eradication rate can be defined as the percentage of patients, who initially are infected with the organism and who have become culture-negative of the organism following an antibiotic treatment. The organism eradication rate is usually expressed as a function of time after the initiation of the antibiotic treatment, where the number of patients with negative culture usually increases with the duration of the treatment. The speed of increase in the number of culture-negative patients may depend on the values of certain pharmacodynamic markers of the antibiotics in the specific patient population.

The values of the pharmacodynamics markers can be further correlated to the antibiotic dose regimens and patient characteristics. The correlation can be described by population pharmacokinetic models.

One particular example demonstrates the application of the present invention in estimating the effective regimens of ciprofloxacin for treating anthrax, and in estimating the survival rate of anthrax-infected patients following various ciprofloxacin regimens. In this example, the effective dose regimen and patient survival rate are estimated based on the method derived from (1) the survival rate of controlled patients receiving inactive treatments, and (2) the correlation between and the organism eradication rate, the pharmacodynamics, and the pharmacodynamics of ciprofloxacin. The method adequately predicts the survival rate of anthrax-infected patients following ciprofloxacin treatment in an anthrax outbreak independent of the source data for deriving the method.

There are several advantages of the present invention over other approaches of prior art. The invention provide methods that directly estimate the survival rates of antibiotic treatments for fatal infectious diseases, and eliminate the potential drawbacks involved in targeting the pharmacodynamic markers alone. While targeting pharmacodynamic markers is better than the one-size-fits-all approach, the invention provides more complete information of patient survival for rational decisions on dose selection of antibiotic treatments for fatal infectious diseases. Further, the invention utilizes survival data from the controlled patients and does not require additional clinical studies in any sub-population, which approach makes the invention economically affordable.

### BRIEF DESCRIPTION OF THE SEVERAL VIEWS OF THE DRAWING

FIG. 1 shows the survival curves of controlled patients in the Sverdlovsk anthrax outbreak in 1979 [Meselson et al, 1994].

FIG. 2 shows that the bacteria eradication rate after 1 to 10 days of ciprofloxacin treatment is a function of AUC/MIC values [Forrest et al, 1993(b)].

FIG. 3 shows the survival curves of patients in the Sverdlovsk anthrax outbreak in 1979 [Meselson et al, 1994], patients in the US bioterrorism attacks in 2001 [CDC MMWR Weekly, 2001], and monkey in the control group of an animal experiment [Friedlander et al, 1993].

FIG. 4 shows the distribution of onset-to-treatment time in the Sverdlovsk [Walker, 2000] and US patients [CDC MMWR Weekly, 2000].

FIG. 5 shows the bacteria eradication rate after 1 to 10 days of ciprofloxacin treatment as a function of AUC/MIC values, independent of the type of organisms [Forrest et al, 1993(b)].

FIG. 6 shows the estimated distribution of AUC/MIC (based on population model described by Equation 10) for a population with CLcr=120 mL/min/1.73 m³ and BW=70 kg, following 400 mg bid ciprofloxacin 1-hr iv infusion.

FIG. 7 shows the estimated overall survival rates on day 10 after disease onset in patients with creatinine clearance equal to 120 mL/min/1.73 m³, body weight ranging from 70 to 150 Kg, following various bid dose regimens of ciprofloxacin initiated from 0 to 5 days after the disease onset.

FIG. 8 shows the estimated overall survival rates on day 10 after disease onset in a typical patient population (BW=70 Kg and Cl_{cr}=120 mL/min/1.73 m³), compared with the historical data. The historical data consist of the overall survival data of the victims in the US bioterrorism attacks (FIG. 1) with the average onset-to-treatment time=4 days (FIG. 2); the overall survival rate of the patients in the Sverdlovsk outbreak (FIG. 1), assuming no effective treatment was given (onset-to-treatment>8 days); the survival rate of monkeys [Friedlander et al, 1993] with comparable ciprofloxacin plasma concentration (treatment initiated on day 0); and the 6-point running average of the US data.

FIG. 9 shows the individual plasma concentration profiles (dashed lines) of two hypothetical subjects, which are determined applying the Bayesian estimation based on observed plasma concentrations and the population pharmacokinetic model (solid lines represent the population-mean concentration profiles).

FIG. 10 shows the overall survival rate on day 10 after disease onset of the patients shown in FIG. 8, as a function of dose regimen and onset-to-treatment time determined by the survival model.

### DETAILED DESCRIPTIONS OF THE INVENTION

The present invention provides new methods for determining the effective antibiotic regimens for treating fatal infectious diseases. The invention provides methods to estimate the patient survival rate following antibiotic treatments for fatal infectious diseases, based on (1) the survival rate of controlled patients receiving no treatment or inactive treatments, and (2) the correlation between organism eradication rate and antibiotic regimen. The invention also provides methods to estimate the effective antibiotic regimens for treating fatal infectious diseases, based on (1) the survival rate of controlled patients receiving no treatment or inactive treatments, (2) the correlation between organism eradication rate and antibiotic regimen, and (3) the targeted therapeutic effect on the survival rate of the antibiotic-treated patients.

A. Fatal Infectious Disease

Numerous infectious diseases are fatal if the patients are not treated immediately with proper antibiotic regimens. A list of fatal infectious diseases is shown in Table 1. The effective antibiotic regimens for treating the diseases may vary significantly among different patient populations, depending on their demographic variables and other characteristics. The present invention provides methods to estimate the patient survival rates and the effective antibiotic regimens for each population without actually conducting clinical studies in the populations.

TABLE 1
A list of fatal infectious diseases.

| Disease | Descriptions | Reference |
|---|---|---|
| Aeromonas | Crude fatality rate within 2 weeks after | Ko et al, 2000 |
| bacteremia | the onset was 30%. | |
| Anthrax | Anthrax is a fatal zoonotic infection that | Meselson et |
| | has been recognized as a human disease | al, 1994 |
| | for thousands of years. | |
| Bacteremia, | Gram-negative bacteremia and ensuing | Spapen, 1993 |
| sepsis | sepsis and septic shock remain a leading | |
| | cause of morbidity and mortality after | |
| | surgery and in critically ill patients. | |
| Bacterial | The bacteriologic cure rate and survival | Cherubin, |
| meningitis | rate of bacterial meningitis were about 85 | 1986 |
| | percent if treated with antibiotics. | |
| Candida | Invasive and disseminated Candida | Dean, 1998 |
| infections | infections have become a major source | |
| | of morbidity and mortality in the modern | |
| | surgical intensive care unit. | |
| Community- | Community-acquired pneumonia are | Bartlett, 2000 |
| acquired | the major cause of death due to infectious | |
| pneumonia | disease in the developed and developing | |
| | world. Pneumonia is the leading cause | |
| | of death due to infectious disease in | |
| | the elderly. | |
| Epiglottitis | Epiglottitis in adults is a dangerous | Kuppens et |
| | infectious disease with a rising incidence | al, 2000 |
| | and potential fatal complications | |
| Fournier's | Fournier's gangrene is a fatal infectious | Ochiai et al, |
| gangrene | disease with necrotic fasciitis of the | 2001 |
| | external genitalia. | |
| Infections in | Infections remain common life-threatening | Engels et al, |
| bone marrow | complications of bone marrow | 1999 |
| trans- | transplantation. | |
| plantation | | |
| Infections in | Infections are a major complication of | Patel et al, |
| solid-organ | solid-organ transplantation. Fungal | 1997 |
| trans- | infections, caused by both yeasts and | |
| plantation | mycelial fungi, are associated with the | |
| | highest mortality rates. | |
| Infective | Infective endocarditis remains a deadly | Chamoun et |
| endocarditis | disease. | al, 2000 |
| Melioidosis | Melioidosis is an infectious disease caused | Samuel et al, |
| | by a bacterium (Burkholderia | 2001; |
| | pseudomallei) found particularly | Maneechotes |
| | in some areas in the tropics. | uwan, 1999 |
| | It is a serious condition which can be fatal. | |
| | Melioidosis is an important disease in | |
| | terms of mortality rate and it requires | |
| | rapid diagnosis and treatment. | |
| Spinal | Spinal infection has a high rates of | Wisneski, |
| infection | morbidity and mortality in the past. | 1991 |

B. Pharmacodynamics Markers of Antibiotics

A number of pharmacodynamics biomarkers for the efficacy of antibiotics have been defined in the literature [Sanchez-Navarro et al, 1999; Hyatt et al, 1995] that consider microbiological and pharmacokinetic parameters together. These biomarkers are intended for evaluating the potential efficacy of antimicrobial treatments that is correlated to the values of the biomarkers. Evidences have demonstrated the benefits of adjusting antibiotic dose regimen based on these biomarkers. The practice of individualizing dose regimen has been successfully applied to the treatments of respiratory and other types of infection [Madaras-Kelly et al, 1996; Hyatt et al, 1994]. The pharmacodynamic biomarkers most studied and recommended as predictors of the response to anti-infective therapies include:

Cmax/MIC: The ratio of the maximum plasma drug concentration to the minimum inhibitory concentration.

AUC/MIC: The ratio of the area under the plasma drug concentration curve to the minimum inhibitory concentration.

Tmic: Time for which the plasma drug concentration exceeds MIC.

AUC_{>mic}: Area under the drug concentration curve for which the concentration exceed MIC.

PK variable: Other pharmacokinetic parameters that are derived from the antibiotic plasma concentration.

Shown in Table 2 is a list of antibiotics of which the effectiveness may be correlated to pharmacodynamic markers

TABLE 2
List of antibiotics of which the effectiveness may be
correlated to pharmacodynamic markers [Craig, 2001;
Schentag et al, 2001; Mouton et al, 1999; Li 2000;
Crokaert, 2001; MacGowan 2001].

| | | Examples of Antibiotics |
|---|---|---|
| | Drug Classes of Which | of Which the |
| | the effectiveness be | effectiveness may be |
| | correlated to the | correlated to the |
| Pharmacodynamic | Pharmacodynamic | Pharmacodynamic |
| Marker | Markers | Markers |
| Cmax/MIC | aminoglycosides, | levofloxacin, ciprofloxacin, |
| | fluoroquinolones, | gemifloxacin, |
| | glycopeptides | Moxifloxacin, |
| | | gatifloxacin, clinafloxacin, |
| | | sparfloxacin, trovafloxacin, |
| | | grepafloxacin, ofloxacin, |
| | | norfloxacin, lomefloxacin, |
| | | fleroxacin, pefloxacin, |
| | | amifloxacin |
| Tmic | β-lactams, macrolides, | Clindamycin, amoxicillin, |
| | linezolid, pennicillins, | erythromycin, |
| | cephalosporines | clarithromycin, tobramycin, |
| | | cefmenoxine, ticarcillin, |
| | | ceftazidime, vancomycin, |
| | | azithromycin, |
| | | roxithromycin, |
| | | oleandomycin, spiramycin, |
| | | josamycin, miocamycin, |
| | | midecamycin, rosaramycin, |
| | | troleandomycin, |
| | | flurithromycin, |
| | | rokitamycin, |
| | | dirithromycin, |
| | | streptomycin, |
| | | gentamycin, amikacin, |
| | | netilmicin, kanamycin, |
| | | neomycin, penicillin G, |
| | | penicillin V, methicillin, |
| | | oxacillin, cloxacillin, |
| | | dicloxacillin, nafcillin, |
| | | ampicillin, carbenicillin, |
| | | mezlocillin, piperacillin, |
| | | Cephalothin, cefazolin, |
| | | cephalexin, cefadroxil, |
| | | cefamandole, cefoxitin, |
| | | cefaclor, cefuroxime, |
| | | loracarbef, cefonicid, |
| | | cefotetan, ceforanide, |
| | | cefotaxime, cefpodoxime, |
| | | ceftizoxime, ceftriaxone, |
| | | cefoperazone, cefazidime, |
| | | cefepime |
| AUC/MIC | Tetracyclines, | Azithromycin, levofloxacin, |
| | glycopeptides, | ciprofloxacin, |
| | fluoroquinolones, | gemifloxacin, |
| | quinuupristin- | tobramycin, cefmenoxine, |
| | dalfopristin, | Moxifloxacin, gatifloxacin, |
| | β-lactams, quinolone, | clinafloxacin, sparfloxacin, |
| | aminoglycosides | trovafloxacin, netlimicin, |
| | | grepafloxacin, teicoplanin, |
| | | chlotetracycline, |
| | | oxytetracycline, |
| | | demeclocycline, |
| | | methacycline, doxycycline, |
| | | minocycline |

C. Survival Rate of Patients Treated with Placebo or Inactive Treatments or Receiving no Treatment

The survival rate is usually defined as the percentage or the ratio of the patients who survive a disease, relative to the total number of patients contracting the disease. The mortality rate is usually defined as the percentage or the ratio of the patients who die from a disease, relative to the total number of patients contracting the disease. The above descriptions are not meant to set a limitation of the definitions of survival rate or mortality rate.

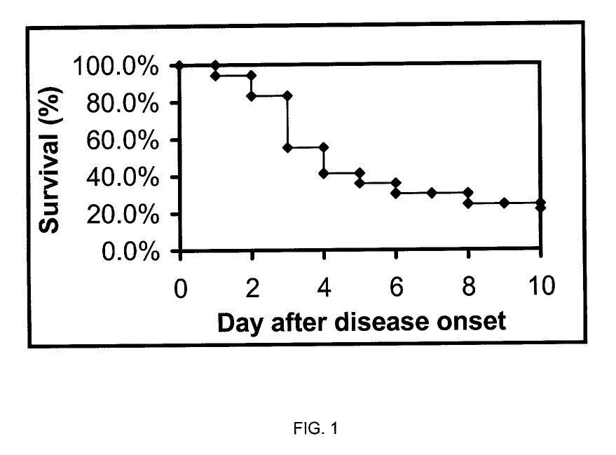

Patients with infectious diseases may sometimes, mostly unintentionally, be treated with inactive treatments (including placebo) or may receive no treatment due to various reasons, such as a lack of available medicines. Although it is unethical to treat patients with inactive treatments, in some exploratory studies, where a new investigative drug is studied, the investigative drug may turn out to be inactive. These patients without a proper treatment for their disease are called the “controlled” patients. The survival data of the controlled patients can be available from epidemiological studies or, in some rare cases, from prospectively designed studies. Shown in FIG. 1 is an example of survival rates of patients who are infected with anthrax and who received ineffective antibiotic treatments.

As shown in FIG. 1, the survival rate of patients is usually calculated as a function of time after the disease onset. The survival rate can be expressed by the following empirical equation:

Survival rate up to day i=Sᵢ% (1)

where day i is the day after the disease onset. Day 0 is the day of disease onset.

The survival rate can also be expressed by the Cox proportional hazard model:

Sₜ%=Exp(−λ*t) (2)

Where Sₜ is the patient survival at time t, λ is an exponent, and t is the time after disease onset.

Equations (1) and (2) are not meant to set a limitation of the types of mathematical descriptions for the patients survival rates.

The mortality rate can be calculated as follow:

Mₜ=1−Sₜ (3)

Where Mₜ is the patient mortality rate on time t. Equation (3) is not meant to set a limitation of the types of mathematical descriptions for the patients mortality rates.

D. Correlation between (1) Organism Eradication Rate and (2) Pharmacodynamic Marker of Antibiotic

The organism eradication rate is usually defined as the percentage or the ratio of the patients that become culture-negative, relative to the initial total number of patients infected with the bacteria. The organism eradication rate usually increases with the duration of an antibiotic treatment. The above description is not meant to set a limitation of the definitions of organism eradication rate. The organism eradication rate can be obtained from human studies [Forrest et al, 1993(b)], animal studies [Firsov et al, 1997], or in vitro studies [McGrath et al, 1994].

A number of pharmacodynamics biomarkers for the efficacy of antibiotics have been defined in the literature [Sanchez-Navarro et al, 1999; Hyatt et al, 1995], which consider microbiological and pharmacokinetic parameters together. These biomarkers are intended for evaluating the potential efficacy of antimicrobial treatments that is correlated to the values of the biomarkers. Evidences have demonstrated the benefits of adjusting antibiotic dose regimen based on these biomarkers. The practice of individualizing dose regimen has been successfully applied to the treatments of respiratory and other types of infection [Madaras-Kelly et al, 1996; Hyatt et al, 1994].

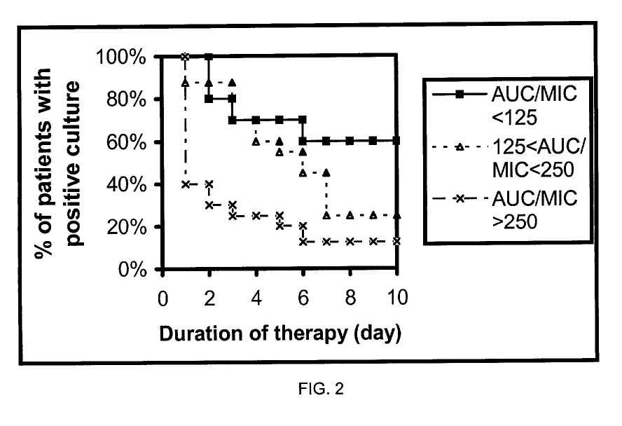

An example of the correlation between organism eradication rate and pharmacodynamic markers is shown in FIG. 2. This example is not meant to set a limitation on the types of correlation between organism eradication rate and pharmacodynamic markers. In this example, patients infected with several gram positive and gram negative bacteria were treated with ciprofloxacin. The relationship between the pharmacodynamic marker, AUC/MIC, of ciprofloxacin and the percentage of patients with blood culture remaining positive on day i after the treatment initiation can be expressed by the following empirical equation:

$$Percentage of culture \text{-} positive patients on day i = {CP}_{< 125 , i} \% if AUC / MIC < 125 \\ Percentage of culture \text{-} positive patients on day i = {CP}_{125 - 250 , i} \% if 125 < AUC / MIC < 250 \\ Percentage of culture \text{-} positive patients on day i = {CP}_{> 250 , i} \% if AUC / MIC > 250 \qquad ( 4 )$$

where day 1 is the first day of treatment.

The relationship between the pharmacodynamic marker and the percentage of patients with blood culture remaining positive on day i after the treatment initiation can also be described by the Cox proportional hazard model:

CPₜ%=Exp(−λ_{cp}*t) (5)

Where CPₜ% is the percentage of culture-positive patients at time t after treatment initiation, and λ_{cp} is the exponent. Further, the exponent, λ_{cp}, is a function of the pharmacodynamic biomarker, AUC/MIC. The exponent, λ_{cp}, can also be a function of dose regimen of ciprofloxacin.

E. Correlation between (1) Pharmacodynamics Marker, and (2) Patient Characteristics and Dose Regimen

The pharmacodynamics markers of an antibiotic are usually functions of patient characteristics and dose regimen. The patient characteristics may include age, gender, body weight, creatinine clearance, and any other demographic variables or biological markers. The correlation can be described by population pharmacokinetic models or other empirical expressions. An example of the population pharmacokinetic model of ciprofloxacin is presented as follows. The example is not meant to set the limitation of the types of the correlation between pharmacodynamic markers, patient characteristics, and dose regimen.

The important covariates affecting the ciprofloxacin pharmacokinetics included body weight and creatinine clearance. The following two-compartment model [Forrest et al, 1993(a)] for iv infusion can be used to describe the population pharmacokinetics of ciprofloxacin:

$$\frac{ⅆ {C}_{1}}{ⅆ t} = - ( {k}_{el} + {k}_{12} ) {C}_{1} + {k}_{21} {C}_{2} , {C}_{1 , i} = {C}_{1} + {ϵ}_{c1 , i \\} \\ \frac{ⅆ {C}_{2}}{ⅆ t} = {k}_{12} {C}_{1} - {k}_{21} {C}_{2} , {C}_{2 , i} = {C}_{2} + {ϵ}_{c2 , i \\} \\ {k}_{el} = \frac{{CL}_{t}}{{V}_{1}} , {K}_{12} = \frac{{CL}_{d}}{{V}_{1}} , {K}_{21} = \frac{C {L}_{d}}{{V}_{2}} \\ {V}_{1} = {V}_{1 , ty} + {η}_{v1} , {V}_{2} = {V}_{2 , ty} + {η}_{v2} , {CL}_{d} = {CL}_{d , ty} + {η}_{cld} , {CL}_{t} = {CL}_{t , ty} + {η}_{clt} \\ {CL}_{t , ty} = ( {θ}_{1} \cdot {CL}_{cr} + {θ}_{2} ) \cdot BW , {V}_{1 , ty} = {θ}_{3} \cdot BW , {V}_{2 , ty} = {θ}_{4} \cdot BW \qquad ( 6 )$$

where C₁ is the plasma concentration, C₂ is the concentration in the peripheral compartment, kₑₗ is the elimination rate constant from the central compartment, k₁₂ and k₂₁ are the distribution rate constants between the central and peripheral compartments, CLₜ is the total clearance, CL_{d} is the distribution clearance, V₁ is the central compartment volume of distribution, V₂ is the peripheral compartment volume of distribution, CL_{cr} is the creatinine clearance of the patient, BW is the body weight of the patient, θ represents the covariate model parameter, η represents the between-subject variability, ε represents the within-subject variability and the residual error of the model, i denotes the individual values, and ty denotes the typical population values.

F. Correlation between Organism Eradication Rate and Antibiotic Regimen

The correlation between organism eradication rate and antibiotic regimen can be derived utilizing one of more of the following information:

1. the pharmacodynamic marker of the antibiotic;

2. the pharmacokinetics of the antibiotic;

3. the characteristic of the patients;

For example, such correlation for ciprofloxacin can be derived from Equations (4) or (5), which contain pharmacodynamic information of the antibiotic, plus Equation (6), which contains the pharmacokinetics and patient characteristics. In some instances, the correlation between pharmacokinetics and patient characteristics may not exist or may not be available. In this case, Equation (6) will not contain the patient characteristics information. In other instances, the correlation between organism eradication rate and antibiotic regimen can be established directly using only the pharmacodynamic information. An example of the last case is Equation (5) for ciprofloxacin where the exponent, λ_{cp}, can be a function of dose regimen of ciprofloxacin.

G. Overall Correlation between Patient Survival and Antibiotic Regimen

The overall correlation between patient survival rate and antibiotic regimen can be derived from the following information:

1. the survival rate or the mortality rate of controlled patients with the same disease, who receive no treatment, or receive placebo or inactive treatment;

2. the correlation between (1) the organism eradication rate of the antibiotic and (2) the dose regimen of the antibiotic;

An example of the survival rate of controlled patients is shown in FIG. 1, and can be expressed in a mathematical form in Equations (1) or (2). In this example, the patient survival rate on day i into the antibiotic treatment, which is initiated on day j after the disease onset, can be determined based on the following

$$\% surviving and culture positive patients in Group k on day i after treatement initiation = {SCP}_{i , k} \% = {P}_{k} \cdot {SCP}_{i - l , k} \frac{{CP}_{i}}{{CP}_{i - l}} \frac{{S}_{i + j}}{{S}_{i + j - 1}} \% death occur in Group k on day i after treatment initiation = {D}_{i , k} \% = {P}_{k} \cdot {SCP}_{i - 1 , k} \frac{{CP}_{i}}{{CP}_{i - l}} \frac{{S}_{i + j - 1} - {S}_{i + j}}{{S}_{i + j - 1}} Overall survivial rate for treatment initiated on day j after disease onset = {S}_{j - 1} - {\sum }_{k} {\sum }_{i} {D}_{i , k} \qquad ( 7 )$$

where S, CP, and P are defined in Equations 1, 4, and 11 respectively, k denotes the patients group defined by AUC/MIC ranges in Equation 4, Cpᵢ/Cpᵢ₋₁, is the ratio of patients remaining culture positive from day i−1 to i after treatment initiation, and Sₗ₊ⱼ/Sᵢ₊ⱼ₋1 is the ratio of culture-positive patients (inactive control) remaining alive from day i+j−1 to i+j after disease onset. The basic assumption for the above equation is that the probability of survival for culture-positive patients on day (i+j) after disease onset is the same as the survival rate of the controlled patients without effective treatment on the same day after disease onset.

H. An Example

The following example is included to demonstrate the embodiments of the invention for one of many applications. Those of skill in the art should, in light of the present disclosure, appreciate that many changes can be made in the specific embodiments that are disclosed and still obtain a like or similar result without departing from the spirit and scope of the invention.

Anthrax and Its Treatments

Anthrax is a zoonotic infection that has been recognized as a human disease for thousands of years. Cutaneous, gastrointestinal, and inhalational forms of infection with Bacillus anthracis have been traditional associated with agricultural or industrial exposures. There have been several documented anthrax outbreaks in the recent history, such as those in Sverdlovsk 1979 [Meselson et al, 1994] and in the US 2001 [CDC MMWR Weekly, 2001]. This example will focus on the most lethal form of anthrax infections—inhalation.

Ciprofloxacin is currently recommended as one of the treatments for anthrax. The effectiveness of the treatment is demonstrated primarily based on an experiment conducted in Rhesus Monkeys [Friedlander et al, 1993; Kelly et al, 1992; Physician's Desk Reference 2001]. In the animal experiment, groups of 10 monkeys were exposed to a lethal aerosol dose of Bacillus anthracis spores. One day after the exposure, the animals were treated with the antibiotic continuously for 30 days. The antibiotic regimen provided sufficient protection to the animals while on therapy. The peak and trough plasma concentrations of the antibiotics were obtained from the animals after multiple doses [Kelly et al, 1992]. The required dose regimens of ciprofloxacin [Physician's Desk Reference, 2001] in human for treating anthrax were estimated so that the regimens will produce similar plasma drug concentration in human to those in the animal experiment.

Patient characteristics is an important factor that influences the effectiveness of ciprofloxacin. It has been reported that the dose regimen of ciprofloxacin should be adjusted based on renal function of the patients for both safety [Physician's Desk Reference, 2001] and efficacy [Forrest, et al, 1993] reasons. Dose adjustment of ciprofloxacin based on body weight is also recommended [Forrest, et al, 1993]. In addition, age- and gender-related changes in pharmacokinetics and pharmacodynamic surrogate markers of ciprofloxacin are reported in the literature [Shah et al, 1995]. Since anthrax is a fatal disease, the dose adjustment based on patient characteristics may have clinically significant impact on the patient survival.

Using the method of the present invention, analyses were conducted to establish the model for predicting the survival rate of patients infected with anthrax as a function of the patient characteristics, the time to treatment initiation after disease onset, and the effective dose regimen of ciprofloxacin. The models were developed based on the existing human pharmacokinetics and pharmacodynamics data of ciprofloxacin, and the survival rate of patients in an anthrax outbreak in Sverdlovsk 1979. The model estimates the percentage of patients with complete bacteria eradication as a function of time after treatment initiation, with the eradication rate stratified by the pharmacodynamic marker, AUC/MIC. The time to bacteria eradication is then correlated to the patient survival rate based on documented human survival data following the historical anthrax outbreak. The pharmacodynamic marker is highly variable among patients, influenced by the patient characteristics, such as body weight, renal function, age, and gender. Another critical factor affecting the survival rate is the onset-to-treatment time. The model adequately predicted the overall survival rate of the victims in the recent bioterrorism attacks in the US 2001.

Survival Rate of Controlled Patients Treated with Placebo or Inactive Treatments or Receiving no Treatment

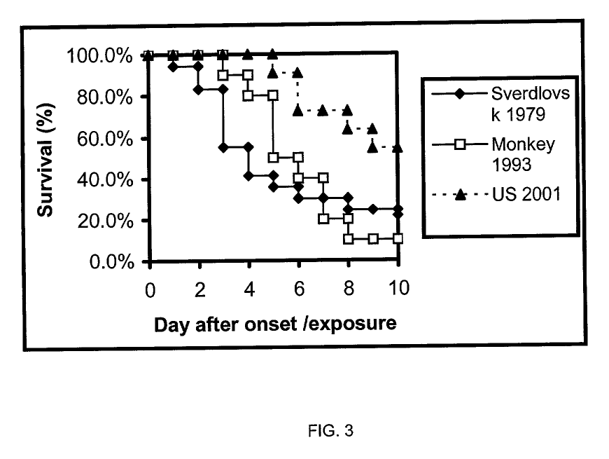

Survival data of human and monkeys exposed to anthrax are available in the literature (FIG. 3). The survival rates of anthrax-exposed Rhesus monkeys were reported [Friedlander et al, 1993] for those treated with 125 mg bid ciprofloxacin and placebo. In this study, groups of 10 monkeys were exposed to a lethal aerosol dose of Bacillus anthracis spores. One day after the exposure, the animals were treated with the antibiotics or placebo continuously for 30 days. The group of animal treated with ciprofloxacin were adequately protected from the disease, while the survival rate was 10% for the control group 10 days after the anthrax exposure (FIG. 3).

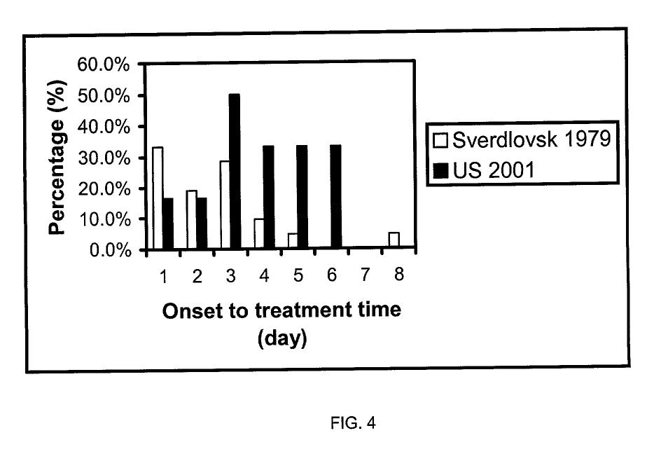

During the Sverdlovsk anthrax outbreak in 1979 [Meselson et al, 1994], there were 79 documented patients infected with inhalational anthrax, and out of these 79 patients, 68 died [Inglesby et al, 1999]. Out of the 68 deaths, 50 had documented onset-to-death time. The survival rates are presented in FIG. 3. The onset-to-treatment time is available in 21 patients [Walker, 2000], and it is plotted in FIG. 4.

There were 11 confirmed inhalational anthrax cases in the bioterrorism attacks in the US from Oct 1^{st} to Nov 30^{th}, 2001 [CDC MMWR Weekly, 2000]. Five out of the 11 patients died before Dec 31. The onset-to-death time is available in all patients [CDC MMWR Weekly, 2000]. The survival rate of these US patients is shown in FIG. 3. The onset-to-treatment time is available in all patients and the distribution is shown in FIG. 4.

It appears that the survival rate of the patients in the Sverdlovsk outbreak was similar to or slightly lower than the survival rate of the monkeys in the control group of the 1993 experiment. The Sverdlovsk patients were reportedly treated with penicillin, cephalosporin, chloramphenicol, anti-anthrax globin, corticosteroids, osmo-regulatory solutions, and artificial respiration. However, the exact dose regimens were not clearly described in the original paper [Meselson et al, 1994]. Out of the 68 deaths in the Sverdlovsk outbreak, 21 had documented onset-to-treatment time (FIG. 4), which did not show any significant treatment delay compared to the US data. The overall mortality rate of the Sverdlovsk patients (86%) is also similar to the occupationally acquired cases in the US (89%) [Inglesby et al, 1999], the later mostly occurring before the advent of antibiotics. Based on the fact that survival rate in the Sverdlovsk outbreak was similar to those of the animal control group and the occupationally acquired cases without antibiotic treatment, it is apparent that the antibiotic treatment given to the Sverdlovsk patients was ineffective. Thus, the survival curve from the Sverdlovsk patients was treated as one obtained from an inactive control group.

The survival rate from the controlled patients can be expressed by the following empirical equation:

Survival rate up to day i=Sᵢ% (8)

where day i is the day after the disease onset, which ranges from 1 to 10 days. Day 0 is the day of disease onset.

The overall survival of the US patients was 55% up to Dec. 31, 2001. This survival rate is significantly higher than that of the Sverdlovsk patients [Meselson et al, 1994], but lower than the ciprofloxacin-treated animals (90% for intend-to-treat) [Friedlander et al, 1993]. It appears that at least ciprofloxacin was given to these patients, perhaps in combination of other antibiotics [CDC MMWR Weekly, 2000].

Correlation between (1) Organism Eradication Rate and (2) Pharmacodynamic Marker of Antibiotic

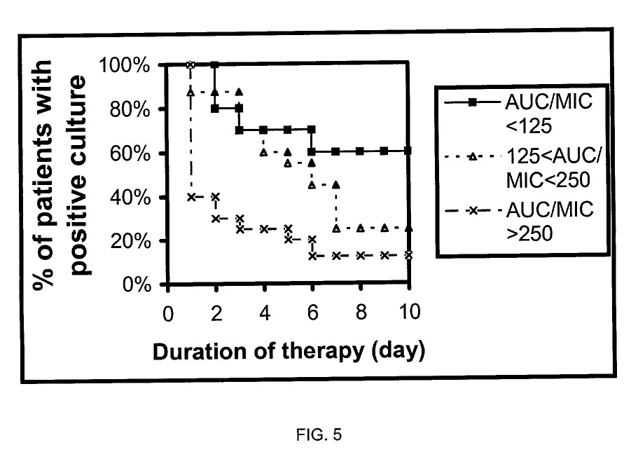

The reported MIC of ciprofloxacin against anthrax varies from 0.06 μg/mL to 0.08 μg/mL [Friedlander et al, 1993; Physician's Desk Reference, 2001]. A mid point of 0.07 μg/mL was adopted in the analyses. It has been demonstrated [Forrest et al, 1993(b); MacGowan et al, 1999; Firsov et al, 1997] that the AUC/MIC values of ciprofloxacin is a good surrogate for bacteria eradication rate and therapeutic outcomes. It is also suggested [Firsov et al, 1998] that the correlation between the antimicrobial effects of ciprofloxacin and AUC/MIC is independent of organism species, gram positive or gram negative. The bacteria eradication rate increases with time and is correlated with the AUC/MIC values (FIG. 5) for both gram positive and negative organisms as shown in a literature study [Forrest et al, 1993(b)], which includes both types of organisms with a MIC range (0.008−0.4 μg/mL) covering that of anthrax. As the patient fatality occurs rapidly after the onset of anthrax, it is critical to treat the patients with the ciprofloxacin regimens producing adequate AUC/MIC values. For the treatment regimens producing AUC/MIC<125, more than 60% of the patients remains culture-positive after 10 days, which is not acceptable for treating the fatal disease, of which the average onset-to-death time is 3 days. Literature data [Forrest et al, 1993(b); Leggett et al, 1989; Craig, 1998; Bedos, 1998] also show that the survival rates of human and animals infected with a variety of gram-positive and gram-negative organisms are correlated to AUC/MIC values of ciprofloxacin and other fluoroquinolones that are used for treating the infections. These literature data indicate that the AUC/MIC values of ciprofloxacin of greater than 100 to 150 are associated with minimum mortality rates.

The relationship between AUC/MIC values and the percentage of patients with blood culture remaining positive on day i after the treatment initiation can be expressed by the following empirical equation:

$$Percentage of culture \text{-} positive patients on day i = {CP}_{< 125 , i} \% if AUC / MIC < 125 \\ Percentage of culture \text{-} positive patients on day i = {CP}_{125 - 250 , i} \% if 125 < AUC / MIC < 250 \\ Percentage of culture \text{-} positive patients on day i = {CP}_{> 250 , i} \% if AUC / MIC > 250 \qquad ( 9 )$$

where day 1 is the first day of treatment.

Correlation between Pharmacodynamics Markers Patient Characteristics, and Dose Regimen

Population pharmacokinetics of ciprofloxacin has been reported in several publications [Forrest et al, 1993(a); Breilh et al, 2001; Terzivanov et al, 1998]. The one-compartment model was proposed for oral regimens, and the two-compartment model was suggested for iv regimens. The important covariates affecting the ciprofloxacin pharmacokinetics included body weight and creatinine clearance. The following two-compartment model [Forrest et al, 1993(a)] for iv infusion was adopted in the analysis:

$$\frac{ⅆ {C}_{1}}{ⅆ t} = - ( {k}_{el} + {k}_{12} ) {C}_{1} + {k}_{21} {C}_{2} , {C}_{1 , i} = {C}_{1} + {ϵ}_{c1 , i \\} \\ \frac{ⅆ {C}_{2}}{ⅆ t} = {k}_{12} {C}_{1} - {k}_{21} {C}_{2} , {C}_{2 , i} = {C}_{2} + {ϵ}_{c2 , i \\} \\ {k}_{el} = \frac{{CL}_{t}}{{V}_{1}} , {K}_{12} = \frac{{CL}_{d}}{{V}_{1}} , {K}_{21} = \frac{C {L}_{d}}{{V}_{2}} \\ {V}_{1} = {V}_{1 , ty} + {η}_{v1} , {V}_{2} = {V}_{2 , ty} + {η}_{v2} , {CL}_{d} = {CL}_{d , ty} + {η}_{cld} , {CL}_{t} = {CL}_{t , ty} + {η}_{clt} \\ {CL}_{t , ty} = ( {θ}_{1} \cdot {CL}_{cr} + {θ}_{2} ) \cdot BW , {V}_{1 , ty} = {θ}_{3} \cdot BW , {V}_{2 , ty} = {θ}_{4} \cdot BW \qquad ( 10 )$$

where C₁ is the plasma concentration, C₂ is the concentration in the peripheral compartment, kₑₗ is the elimination rate constant from the central compartment, k₁₂ and k₂₁ are the distribution rate constants between the central and peripheral compartments, CLₜ is the total clearance, CL_{d} is the distribution clearance, V₁ is the central compartment volume of distribution, V₂ is the peripheral compartment volume of distribution, CL_{cr} is the creatinine clearance of the patient, BW is the body weight of the patient, θ represents the covariate model parameter, η represents the between-subject variability, ε represents the within-subject variability and the residual error of the model, i denotes the individual values, and ty denotes the typical population values.

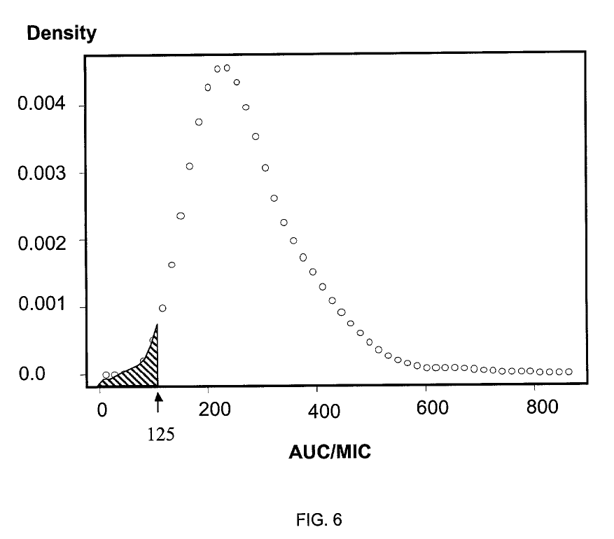

The population-mean of a pharmacodynamic marker, such as AUC/MIC, can be estimated from Equation 10, given the creatinine clearance and body weight of the population. The population-distribution of the pharmacodynamic marker in the typical population can also be estimated, based on the between-subject and within-subject variability. The Monte-Carlo simulation technique [Lee, 2001] can be used to estimate the population distribution of the pharmacodynamic marker. An example of the population distribution of AUC/MIC is shown in FIG. 6, where the patient population with creatinine clearance=120 mL/min/1.73 m³ and body weight=70 Kg is given 400 mg bid ciprofloxacin 1-hr iv infusion. The distribution of the steady-state pharmacodynamic marker AUC₂₄ₕ/MIC for 1000 such patients is shown in the plot. The percentage of the patients with AUC/MIC<125 following the dose regimen can be calculated as the ratio of the area under the curve where AUC/MIC<125 (the shaded area in the plot) to the total area under the distribution curve. Similarly, the percentages of the patients with 125<AUC/MIC<250 orAUC/MIC>250 can be estimated from the distribution profile in FIG. 6. The patient distribution with various AUC/MIC values can be expressed by the following empirical equation:

$$Percentage of patients with AUC / MIC < 125 = {P}_{AUC / MIC < 125} \% \\ Percentage of patients with 125 < AUC / MIC < 250 = {P}_{125 < AUC / MIC < 125} \% \\ Percentage of patients with AUC / MIC > 250 = {P}_{AUC / MIC > 125} \% \qquad ( 11 )$$

The percentage of patients with positive culture after the treatment initiation for patient groups with different AUC/MIC is shown in FIG. 5. The overall percentage of patients with positive culture after a specific time, day i, following the treatment initiation can then be calculated as follows:

$$\% patients with positive culture on day i = {\sum }_{j = 1 , 3} {P}_{j} \cdot {CP}_{j , i} \qquad ( 12 )$$

where j=1 to 3, corresponding to AUC/MIC<125, 125<AUC/MIC<250, and AUC/MIC>250 respectively, and CP and P are defined in Equations (9) and (11).

Correlation between Patient Survival, Organism Eradication Rate, and Antibiotic Regimen

The probability of patient survival on day i into the treatment, which is initiated on day j after the disease onset, will be determined based on the following Equation:

$$\begin{matrix}\% surviving and culture positive patients in Group k on day i after treatement initiation = {SCP}_{i , k} \% = {P}_{k} \cdot {SCP}_{i - l , k} \frac{{CP}_{i}}{{CP}_{i - l}} \frac{{S}_{i + j}}{{S}_{i + j - 1}} \% death occur in Group k on day i after treatment initiation = {D}_{i , k} \% = {P}_{k} \cdot {SCP}_{i - 1 , k} \frac{{CP}_{i}}{{CP}_{i - l}} \frac{{S}_{i + j - 1} - {S}_{i + j}}{{S}_{i + j - 1}} & ( 13 )\end{matrix} Overall survivial rate for treatment initiated on day j after disease onset = {S}_{j - 1} - {\sum }_{k} {\sum }_{i} {D}_{i , k}$$

where S, CP, and P are defined in Equations 8, 9, and 11 respectively, k denotes the patients group defined by AUC/MIC ranges in Equations 9 and 11, Cpᵢ/Cpᵢ₋₁ is the ratio of patients remaining culture positive from day i−1 to i after treatment initiation, and Sₗ₊ⱼ/Sᵢ₊ⱼ₋₁ is the ratio of culture-positive patients (inactive control) remaining alive from day i+j−1 to i+j after disease onset. The basic assumption for the above equation is that the probability of survival for a culture-positive patient on day (i+j) after disease onset is the same as the survival rate of the control group without effective treatment on the same day after disease onset. The assumption is based on the observation that even a small amount of bacteria level (10 cfu/mL) [Friedlander et al, 1993] may cause death in animals. Bacteremia at levels of 10-10⁵ cfu/mL was present in the control monkeys before their deaths due to anthrax [Friedlander et al, 1993].

Survival Rate by Patient Characteristics, Dose Regimens, and Onset-to-Treatment Time

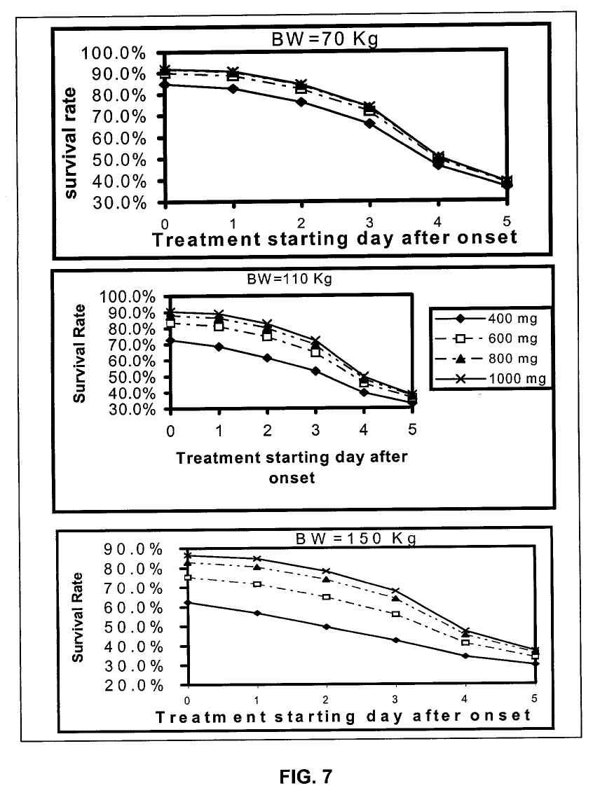

Based on the overall survival model described above (Equations 8-13), the survival rates of anthrax-exposed patients on day 10 after disease onset were estimated as a function of patients characteristics and treatment initiation days (FIG. 7). Survival rates of three groups of patients were included in the analysis, with body weight equal to 70 Kg, 110 Kg, and 150 Kg respectively, and creatinine clearance equal to 120 mL/min/1.73 m³. Four different dose regimens of ciprofloxacin were given to the patients: 400, 600, 800, and 1000 mg bid 1-hr iv infusion. The overall survival rate on day 10 after disease onset was estimated as a function of the onset-to-treatment time.

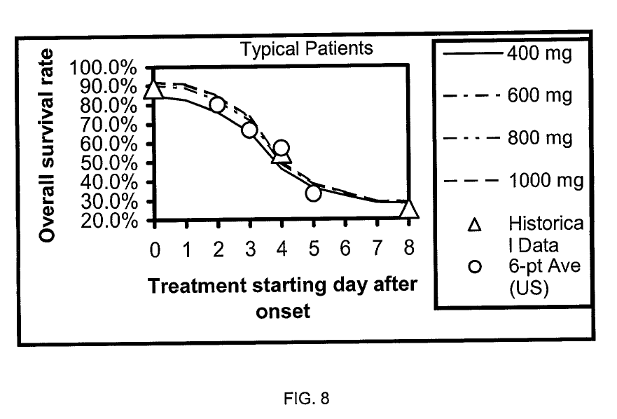

The results show that the 70-kg group slightly benefits by an increase in dose from 400 mg to 600 mg bid, while raising the dose further does not increase the survival rate. The 110-Kg group continuously benefits from increasing dose regimens, with the mortality rate drops by 3 folds from 30% following 400 mg bid to 10% following 1000 mg bid regimens. The increase in the survival rate of the 150-kg group with increasing dose is even dramatic. The result also indicates a trend of reducing overall survival rate with increasing onset-to-treatment time. The severely ill patients with long onset-to-treatment time may particularly benefit from high ciprofloxacin doses. The estimated survival rates in a typical patient population (BW=70 Kg and Cl_{cr}=120 mL/min/1.73 m³) are consistent with the historical data (FIG. 8).

Drug Monitoring

Monitoring of antibiotic treatments for anthrax is highly justifiable, as the disease is fatal, the treatment period can be significantly long that provides opportunities for dose adjustment, and the pharmacokinetics and pharmacodynamics are quite variable among patients. Drug monitoring data can be used to fine-tune individualized dose regimen of ciprofloxacin treatment for anthrax. The pharmacokinetic model parameters in the individual patient can be determined applying the Bayesian estimation, based on the monitoring plasma concentrations in the patient, C_{obs}, and the population-mean model parameters, θₚₒₚ. Subsequently, the individual pharmacodynamic markers are determined using the Bayesian estimate of the individual pharmacokinetic model. The following equation describes the objective function of the Bayesian estimation:

$$Obj {\sum }_{i} \frac{{( {C}_{obs , i} - {C}_{i} ( {θ}_{pred} , {t}_{i} ) )}^{2}}{{σ}_{c}} + {\sum }_{j} \frac{{( {θ}_{pred , j} - {θ}_{pop , {j}_{i}} )}^{2}}{{σ}_{θ {,}_{j}}} \qquad ( 14 )$$

where O_{pred,j} is the j-th predicted model parameter in Equation 10, and Cᵢ (θ_{pred}, tᵢ) is the i-th predicted plasma concentration at time tᵢ, σ_{c} is the variance of concentration, and σ_{θ,j} is the variance of the parameter θⱼ.

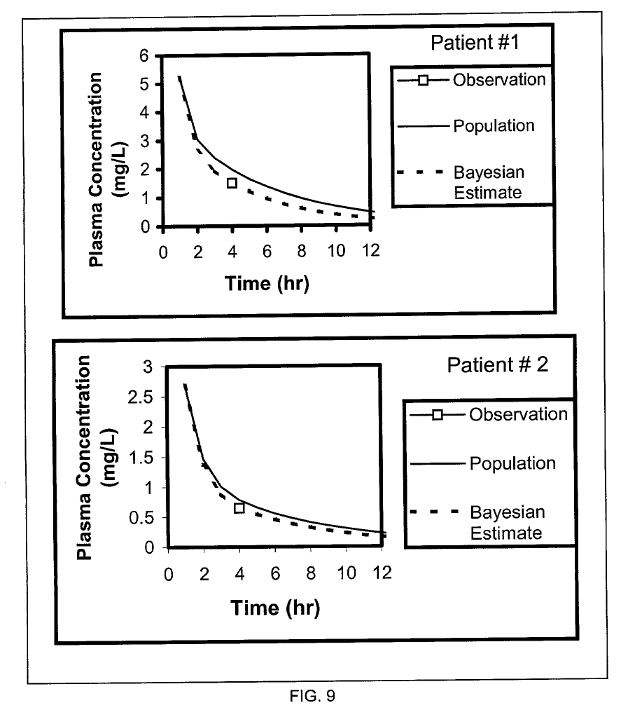

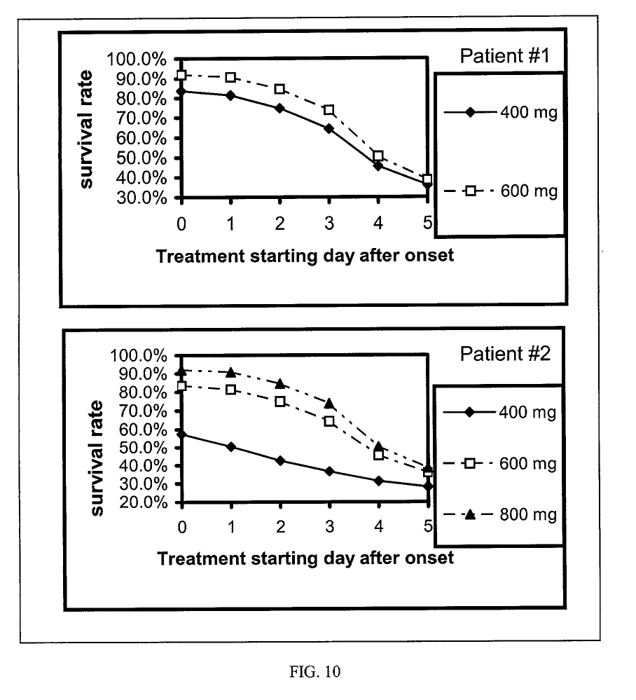

An analysis is conducted to illustrate the utility of drug monitoring in treating anthrax. Two hypothetical anthrax-exposed patients, with a creatinine clearance of 120 mL/min/1.73 m³ and body weight of 70 and 150 Kg respectively, are analyzed. Initially, both patients are given 400 mg bid ciprofloxacin iv infusion. Four hours following the first dose, the patients achieve a plasma concentration of 1.5 and 0.65 mg/L respectively. Applying the Bayesian estimation, the full pharmacokinetic model in each patient is estimated based on the drug monitoring data and the population pharmacokinetic model (Equation 10) by minimizing the objective function (Equation 14). The estimated concentration full profiles following the first dose in the two patients are shown in FIG. 9. It is apparent that patient #2 has a lower (one-half) plasma concentration of ciprofloxacin than patient # 1 due to the difference in body weight. With the estimated pharmacokinetic model for each patient, the overall survival rates (FIG. 10) in the patients as a function of dose regimen and onset-to-treatment time are determined with the survival model. The survival rate of Patient # 1 slightly increases by increasing ciprofloxacin dose from 400 to 600 mg bid. On the other hand, the mortality rate (1—survival rate) of Patient #2 can be reduced by as much as 4 folds if the dose is increased from 400 mg to 800 mg bid. Note that, while the dose is doubled in patient # 2, the plasma concentrations of ciprofloxacin in the two patients differ only slightly, due to the larger body weight of patient #2. Therefore, the probability of toxicity caused by ciprofloxacin in the two patients is not directly correlated to the dose given.

Which is claimed is:

1. Methods for estimating the survival rate or mortality rate of patients with a fatal infectious disease following an antibiotic treatment, said methods comprising:
(a) determining the survival rate or the mortality rate of controlled patients with the same disease, said controlled patients receiving no treatment, or receiving placebo or an inactive treatment;
(b) determining a correlation between (1) the organism eradication rate of said antibiotic and (2) the dose regimen of said antibiotic;
(c) estimating the survival rate or the mortality rate of the antibiotic-treated patients based on (1) the survival rate or the mortality rate of the controlled patients, and (2) the organism eradication rate.

2. Methods for providing evidence to support an effective dosing regimen of an antibiotic for treating a fatal infectious disease using the estimated survival rate or mortality rate of said patients, said methods comprising:
(a) determining the survival rate or the mortality rate of controlled patients with the same disease, said controlled patients receiving no treatment, or receiving placebo or an inactive treatment;
(b) determining a correlation between (1) the organism eradication rate of said antibiotic and (2) the dose regimen of said antibiotic;
(c) estimating the survival rate or the mortality rate of the antibiotic-treated patients based on (1) the survival rate or the mortality rate of the controlled patients, and (2) the organism eradication rate;
(d) determining the effective dose regimen of said antibiotic by targeting the survival rate or mortality rate of the antibiotic-treated patients.

3. The method of anyone of claims 1 and 2, wherein said correlation between the organism eradication rate and the antibiotic dose regimen is derived utilizing one or more of:
(a) the pharmacodynamic marker of said antibiotic;
(b) the pharmacokinetics of said antibiotic;
(c) the characteristic of said patients.

4. The method of anyone of claims 1 and 2, wherein said disease is Aeromonas bacteraemia, Anthrax, Bacteremia, sepsis, Bacterial meningitis, Candida infections, Community-acquired pneumonia, Epiglottitis, Fournier's gangrene, Infections in bone marrow transplantation, Infections in solid-organ transplantation, Infective endocarditis, Melioidosis, or Spinal infection.

5. The method of anyone of claims 1 and 2, wherein said antibiotic is a aminoglycosides, fluoroquinolones, glycopeptides, β-lactams, macrolides, linezolid, pennicillins, cephalosporines, tetracyclines, quinuupristin-dalfopristin, or quinolone.

6. The method of anyone of claims 1 and 2, wherein said antibiotic is amifloxacin, amikacin, amoxicillin, ampicillin, azithromycin, carbenicillin, cefaclor, cefadroxil, cefamandole, cefazidime, cefazolin, cefepime, cefinenoxine, cefonicid, cefoperazone, ceforanide, cefotaxime, cefotetan, cefoxitin, cefpodoxime, ceftazidime, ceftizoxime, ceftriaxone, cefuroxime, cephalexin, Cephalothin, chlotetracycline, ciprofloxacin, clarithromycin, clinafloxacin, cloxacillin, demeclocycline, dicloxacillin, dirithromycin, doxycycline, erythromycin, fleroxacin, flurithromycin, gatifloxacin, gemifloxacin, gentamycin, grepafloxacin, josamycin, kanamycin, levofloxacin, lomefloxacin, loracarbef, methacycline, methicillin, meziocillin, midecamycin, minocycline, miocamycin, moxifloxacin, nafcillin, neomycin, netilmicin, netlimicin, norfloxacin, ofloxacin, oleandomycin, oxacillin, oxytetracycline, pefloxacin, penicillin G, penicillin V, piperacillin, rokitamycin, rosaramycin, roxithromycin, sparfloxacin, sparfloxacin, spiramycin, streptomycin, teicoplanin, ticarcillin, tobramycin, tobramycin, troleandomycin, trovafloxacin, trovafloxacin, or vancomycin.

7. The method of anyone of claims 1 and 2, wherein said organism eradication rate is the one against the organisms causing the said disease or the one against similar organisms susceptible to said antibiotic.

8. The method of anyone of claims 1 and 2, wherein said organism eradication rate is obtained from human, animal, or in vitro studies.

9. The method of anyone of claims 1 and 2, wherein said organism eradication rate reflects time-dependency of the percentage or the ratio of the patients with a positive culture of the organisms, relative to the total number of patients initially infected with the organisms.

10. The method of anyone of claims 1 and 2, wherein said pharmacodynamic marker is one of or a combination of the following:
(a) Cmax/MIC: The ratio of the maximum plasma drug concentration to the minimum inhibitory concentration;
(b) AUC/MIC: The ratio of the area under the plasma drug concentration curve to the minimum inhibitory concentration;
(c) Tmic: Time for which the plasma drug concentration exceeds MIC;
(d) AUC_{>mic}: Area under the drug concentration curve for which the concentration exceed MIC;
(e) PK parameters: Pharmacokinetic parameters that are derived from the antibiotic plasma concentration.

11. The method of anyone of claims 1 and 2, wherein said characteristic of said patients is one of or a combination of the following: age, body weight, gender, body surface areas, and creatinine clearance.

12. The method of anyone of claims 1 and 2, wherein said characteristic of said patients is one or a combination of: demographic variables, blood chemistry parameters, physiological biomarkers, or pathological biomarkers.

13. The method of anyone of claims 1 and 2, wherein the estimated survival rate or the estimated mortality rate in antibiotic-treated patients are verified by comparing the estimation with the observed survival rate or the observed mortality rate in antibiotic-treated patient patients.

14. The method of anyone of claims 1 and 2, wherein said correlation consists of one or more mathematical equations, statistical equations, or empirical expressions.

15. The method of anyone of claims 1 and 2, wherein said effective dose regimen is a dose regimen that results in increases in patient survival rate or decreases in patient mortality rate.
